(19) United States
(12) Patent Application Publication
(10) Pub. No.: US 2017/0059573 A1
(43) Pub. Date: Mar. 2, 2017

(54) METHOD OF DETECTING PEROXYNITRITE USING A COMPLEX OF A SACCHARIDE AND AN ARYLBORONATE-BASED FLUORESCENT PROBE

(71) Applicant: UNIVERSITY OF BATH, Claverton Down, Bath (GB)

(72) Inventors: Tony D. JAMES, BATH (GB); Steven D BULL, BATH (GB); Xiaolong SUN, BATH (GB)

(73) Assignee: UNIVERSITY OF BATH, Claverton Down, Bath (GB)

(21) Appl. No.: 15/120,971

(22) PCT Filed: Feb. 24, 2015
(86) PCT No.: PCT/GB2015/050513

§ 371 Date: Aug. 23, 2016

(30) Foreign Application Priority Data
Feb. 27, 2014 (GB) 1403470.6

(51) Int. Cl. G01N 33/58; C07F 5/02; C07H 23/00; G01N 33/50

(52) U.S. Cl. CPC G01N 33/582; G01N 33/502; C07F 5/025; C07H 23/00

(57) ABSTRACT

A method of detecting peroxynitrite in a sample is described comprising the steps of: (a) providing a complex of a saccharide with an aryl boronate compound of formula (I): Fp-L¹-Z-L²-Ar—B(OH)₂ (I) wherein: Fp comprises a fluorophore; L¹ and L² are linker groups; Z is a fluorescence switch; and Ar is optionally substituted aryl; (b) contacting said aryl boronate-saccharide complex with said sample, whereby peroxynitrite in said sample cleaves said aryl boronate-saccharide complex to produce a compound of formula (II): F-L¹-Z-L²-Ar—OH (II); and (c) detecting a decrease in a fluorescence intensity of said fluorophore resulting from said cleavage reaction in step (b). Peroxynitrite reacts quantitatively, rapidly, and selectively in step (b) of the reaction, whereby medical conditions associated with elevated peroxynitrite can be diagnosed. Also provided are compounds of formula (I) for use in the methods.

### FIELD OF THE INVENTION

The present invention relates to methods of detecting and measuring peroxynitrite in biological samples and cells, and to the use of such methods in diagnosis.

### BACKGROUND OF THE INVENTION

Peroxynitrite (ONOO⁻)—a combination of nitric oxide and superoxide radical anion—has been recognized as a strong oxidant in physiological and pathological processes. It was first discovered as a biological endogenous oxidant in 1990. Under physiological conditions, peroxynitrite is also a highly reactive molecule with a very short lifetime (˜10 ms) involved in cell signal transduction and apoptosis in HL-60 cells, and PC-12 cells. Many biomolecules are oxidized and/or nitrated by peroxynitrite-derived radicals, including DNA, tyrosine residues, thiols, and unsaturated fatty-acid-containing phospholipids. Endogenous peroxynitrite formation and/or protein nitration in cardiac and vascular diseases has been implicated in Alzheimer's disease, Parkinson's disease, Huntington's disease, amyotrophic lateral sclerosis, viral myocarditis, septic shock, cardiac allograft, transplant coronary artery disease, idiopathic dilated cardiomyopathy, atrial fibrillation, hypercholesterolemia, atherosclerosis, hypertension, diabetes (including Type 1 diabetes mellitus and Type 2 diabetes mellitus), diabetic nephropathy, and traumatic brain injury. Recently, peroxynitrite was found as a key trigger of skeletal muscle hypertrophy via activation of calcium signalling. Thus, the importance of peroxynitrite has led to researchers to seek effective approaches for its detection.

Synthetic fluorescence probes are powerful tools for peroxynitrite detection since they can measure intracellular ONOO⁻ directly (Chen, X.; Tian, X.; Shin, I.; Yoon, J. Chem. Soc. Rev. 2011, 40, 4783). Working towards the fluorescence detection of peroxynitrite, Yang et al. developed a range of chemo-sensors in which ONOO⁻ reacts with activated ketones to form dioxiranes (Yang, D.; Wang, H.-L.; Sun, Z.-N.; Chung, N.-W.; Shen, J.-G. J. Am. Chem. Soc. 2006, 128, 6004; Sun, Z.-N.; Wang, H.-L.; Liu, F.-Q.; Chen, Y.; Tam, P. K. H.; Yang, D Org. Lett. 2009, 11, 1887; Peng, T.; Yang, D. Org. Lett. 2010, 12, 4932). A three-channel fluorescence probe capable of distinguishing peroxynitrite from hypochlorite has also been designed (Zhang, Q.; Zhu, Z.; Zheng, Y.; Cheng, J.; Zhang, N.; Long, Y. T.; Zheng, J.; Qian, X.; Yang, Y. J. Am. Chem. Soc. 2012, 134, 18479). Recently, Ai et al. reported a genetically encoded fluorescence probe for the detection of peroxynitrite (Chen, Z. J.; Ren, W.; Wright, Q. E.; Ai, H. W. J. Am. Chem. Soc. 2013, 135, 14940). However, it still remains a great challenge to use small-molecular fluorescence probes to detect ONOO⁻ selectively and sensitively amongst the large number of biologically relevant reactive oxygen and nitrogen species. (eg. H₂O₂ and ClO⁻).

It is known that saccharides bind to arylboronate compounds to form stable complexes. This reaction has been used as the basis for fluorometric measurement of blood sugar levels by attaching a fluorophore F to the arylboronate:

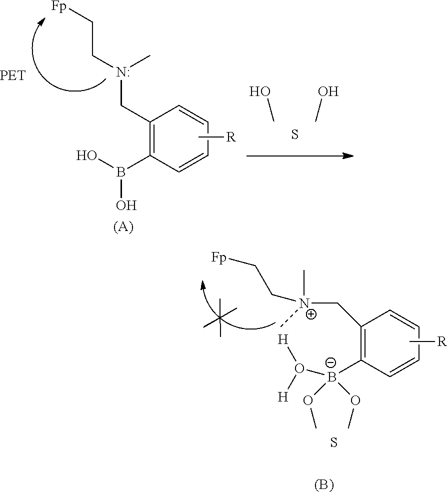

Compound (A) represents a typical arylboronate compound used for detection of saccharides. The fluorophore group Fp in (A) is conjugated to the optionally substituted arylboronate through a tertiary amine group. The fluorescence of the fluorophore is quenched in the free compound due to photo-induced electron transfer (PET) from the tertiary amine nitrogen in the linker group.

In the presence of a saccharide (S) the boronate bonds to two hydroxyl groups on the saccharide ring to form complex (B) in which PET is suppressed, resulting in higher fluorescence intensity from the fluorophore Fp.

A large number of compounds of general formula (A) and related structures, including structures having two arylboronate groups conjugated to a single fluorophore, have been described in the literature for fluorimetric blood glucose monitoring and similar applications. Such compounds are described, for example, in U.S. Pat. No. 5,503,770, US2002115096 and US2008145944.

### SUMMARY OF THE INVENTION

Peroxynitrite is a strong nucleophile. The present inventors have found that peroxynitrite reacts rapidly and stoichiometrically with arylboronate-saccharide complexes of Formula (B) to produce the corresponding aryl alcohol, nitrogen dioxide and a saccharide boronate, according to the following scheme:

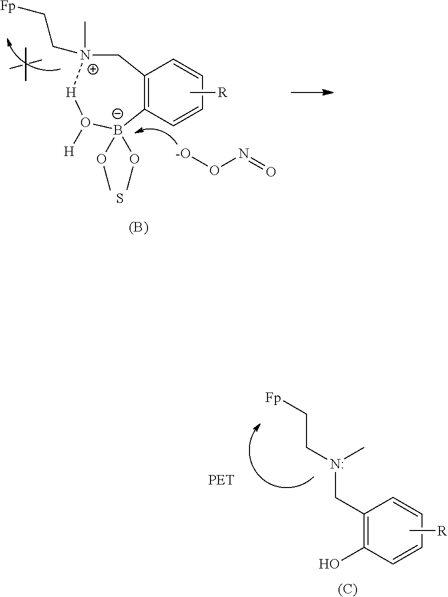

The above reaction releases the tertiary amine group of the linker and thereby restores the PET quenching of the fluorescence of the fluorophore Fp in the product compound (C). The present inventors have further found that other reactive oxygen species and reactive nitrogen species react slowly or not at all with the complex (B) due to protection by internal B—N interaction. Advantageously, this reaction provides a simple fluorometric method for selectively and sensitively identifying and assaying peroxynitrite in the presence of other ROS and RNS, for example in biological systems.

Accordingly, in a first aspect, the present invention provides a method of detecting peroxynitrite in a sample comprising the steps of: (a) providing a complex of a saccharide with an aryl boronate compound of formula (I):

Fp-L¹-Z-L²-Ar—B(OH)₂ (I)
wherein:
Fp comprises a fluorophore;
L¹ and L² are linker groups;
Z is a fluorescence switch group; and
Ar is optionally substituted aryl;
   (b) contacting said aryl boronate-saccharide complex with said sample, whereby peroxynitrite in said sample cleaves said boronate-saccharide complex to produce a compound of formula (II):

Fp-L¹-Z-L²-Ar—OH (II)

and (c) detecting a decrease in a fluorescence intensity of said fluorophore resulting from said cleavage reaction in step (b).

Suitably, the method comprises quantitating an amount of peroxynitrite in the sample by comparing the decrease in fluorescence intensity with decreases observed for reference amounts or concentrations of peroxynitrite.

Suitably, the method is performed on a sample of a biological fluid or tissue, for example a sample removed from a human or animal body. In embodiments, the method further comprises comparing the decrease in fluorescence intensity with a threshold value indicative of a disease state.

In embodiments, the method is performed to detect peroxynitrite in a living cell or in extracellular fluid. In these and other embodiments, the method may be used to image or visualise a spatial distribution of peroxynitrite.

In another aspect, there is provided a kit for detecting peroxynitrite by a method according to the invention, comprising either (i) an arylboronate of Formula (I) and a saccharide in separately packaged form, or (ii) a complex of an arylboronate of Formula (I) and a saccharide in packaged form.

In another aspect, there is provided an arylboronate of Formula (I) and a saccharide for combined use in a method according to the invention.

In another aspect, there is provided an arylboronate of Formula (I), or a complex of an arylboronate of Formula (I) and a saccharide, for use in a method according to the present invention.

In another aspect, there is provided an arylboronate of Formula (I), or a complex of an arylboronate of Formula (I) and a saccharide, for use in a method of diagnosis of a disease state comprising the steps of: measuring the level of peroxynitrite in a sample of biological fluid or tissue removed from the human or animal body by a method according to the present invention, and comparing said level with a reference level of peroxynitrite. For example, the disease state may be selected from any of the disease states related to endogenous peroxynitrite production identified above, for example Alzheimer's disease, Parkinson's disease, Huntington's disease, traumatic brain injury, and skeletal muscle hypertrophy. By way of further example, the disease state may be selected from Alzheimer's disease, Parkinson's disease, Huntington's disease, amyotrophic lateral sclerosis, viral myocarditis, septic shock, cardiac allograft, transplant coronary artery disease, idiopathic dilated cardiomyopathy, atrial fibrillation, hypercholesterolemia, atherosclerosis, hypertension, diabetes (including Type 1 diabetes mellitus and Type 2 diabetes mellitus), diabetic nephropathy, traumatic brain injury and skeletal muscle hypertrophy.

In another aspect, there is provided a method for diagnosing and/or detecting a disease in a subject, comprising the steps of: (i) providing a sample from a subject; (ii) measuring a level of peroxynitrite in said sample according to the method of the present invention; (iii) comparing the measurement from step (ii) with a reference standard; and (iv) using said comparison from step (iii) to determine whether the subject has a disease; (v) optionally, treating said disease; (vi) optionally, repeating steps (i) to (iv) to monitor the progress of the disease.

In another aspect, there is provided a method, a kit or a compound as defined herein with reference to the accompanying description and drawings.

Any feature that is described herein as suitable or preferred in respect of any one aspect of the invention is correspondingly suitable or preferred in respect of all other aspects of the invention.

### BRIEF DESCRIPTION OF THE DRAWINGS

FIG. 1 shows the overall reaction scheme of probe 1 with saccharide and subsequently with peroxynitrite that forms the basis of the methods of the invention.

FIG. 2 shows pH titration of the fluorescence intensity of probe 1 (2 μM) and 1-D-fructose (probe 1, 2 μM; D-fructose, 100 mM), modulating by utilizing aqueous hydrochloric acid (1.2 N) and sodium hydroxide solution (1 N);

FIG. 3 shows. (a) Fluorescence spectra of probe 1 (2 μM) and then addition of D-fructose (100 mM). After addition of D-fructose, the mixtures were stirred for 10 min; (b) Probe 1-D-fructose complex (probe 1, 2 μM; D-fructose, 100 mM) in different concentration of ONOO⁻. After addition of peroxynitrite, the mixtures were stirred for 5 min;

FIG. 4 shows the non-linear relationship between probe 1 (2 μM) and 1-D-fructose (probe 1, 2 μM; D-fructose, 100 mM) fluorescence quenching by different amounts of ONOO⁻ (0-297 μM) at pH 7.3 PBS buffer solution. The spectra were collected after 5 min stirring for each dose with excitation at 410 nm (Ex slit: 5 nm, Em slit: 5 nm);

FIG. 5 shows (a) Fluorescence spectra of free probe 1 (2 μM) in the presence of various peroxynitrite solutions in PBS buffer at pH 7.3; (b) Non-linear relationship of fluorescence quenching of free probe 1 (2 μM) by different concentrations of ONOO⁻ (0-60 μM) at pH 7.3 PBS buffer solution. The spectra were collected after 5 min stirring for each dose with excitation at 410 nm (Ex slit: 5 nm, Em slit: 5 nm);

FIG. 6 shows time-course kinetic measurement of the fluorescence response of probe 1-D-fructose (probe 1, 2 μM; D-fructose, 100 mM) to peroxynitrite (100 μM) at pH 7.30 buffer solution;

FIG. 7 shows (a) Fluorescence spectra of probe 1 after reaction with H₂O₂ in pH 7.30 and pH 8.10. The pH was adjusted from 7.30 to 8.10 using aqueous sodium hydroxide (10 N); (b) Fluorescence spectra of probe 1-D-fructose complex (probe 1, 2 μM; D-fructose, 100 mM) in the presence of hydrogen peroxide (1 mM) at pH 7.3 buffer solution. The spectra were collected at increasing reaction times with excitation at 410 nm (Ex slit: 5.0, Em slit: 5.0);

FIG. 8 shows fluorescence spectra of probe 1-D-fructose (probe 1, 2 μM; D-fructose, 100 mM) in the presence of various ROS/RNS: ONOO⁻ (100 μM, 5 min), ⁻OCl (100 μM, 1 h), H₂O₂ (1 mM, 1 h), NO₂⁻ (1 mM, 1 h), NO₃⁻ (1 mM, 1 h), ROO. (1 mM, 1 h), .O₂⁻ (100 μM, 1 h), .OH (100 μM, 1 h), NO (100 μM, 1 h) at pH 7.30 buffer solution;

FIG. 9 shows relative total changes in fluorescence intensity of various ROS/RNS at pH 7.3 buffer solution with probe 1-D-fructose complex (probe 1, 2 μM; D-fructose, 100 mM).

### DETAILED DESCRIPTION OF THE INVENTION

In the following description, the following definitions apply unless otherwise stated:

Alkyl refers to a branched or unbranched saturated hydrocarbyl radical. Suitably, the alkyl group comprises from about 3 to about 30 carbon atoms, for example from about 5 to about 25 carbon atoms.

Alkylene refers to a corresponding diradical, for example used as or in a linker group.

Alkenyl refers to a branched or unbranched hydrocarbyl radical containing one or more carbon-carbon double bonds. Suitably, the alkenyl group comprises from about 3 to about 30 carbon atoms, for example from about 5 to about 25 carbon atoms.

Alkenylene refers to a corresponding diradical for example used as or in a linker group.

Halogen refers to fluorine, chlorine, bromine or iodine, preferably fluorine or chlorine.

Cycloalkyl refers to an alicyclic moiety, suitably having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

Aryl refers to an aromatic ring system comprising 6, 7, 8, 9, 10, 11, 12, 13, 14, 15 or 16 ring carbon atoms. Aryl may be a polycyclic ring system, having two or more rings, at least one of which is aromatic. This term includes reference to groups such as phenyl, naphthyl, fluorenyl, azulenyl, indenyl, anthryl and the like.

The prefix (hetero) herein signifies that one or more of the carbon atoms of the group may optionally be substituted by nitrogen, oxygen, phosphorus, silicon or sulfur. Heteroalkyl groups include for example, alkyloxy groups and alkythio groups. Heterocycloalkyl or heteroaryl groups herein may have from 3, 4, 5, 6, 7, 8, 9, 10, 11, 12, 13, 14, 15 or 16 ring atoms, at least one of which is selected from nitrogen, oxygen, phosphorus, silicon and sulfur. In particular, a 3- to 10-membered ring or ring system and more particularly a 5- or 6-membered ring, which may be saturated or unsaturated. For example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, 1,3-Dioxo-1,3-dihydro-isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, [beta]-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, 3,4-dihydro-2H-isoquinolin-1-one, 3,4-dihydro-2H-isoquinolinyl, and the like.

“Substituted” signifies that one or more, especially up to 5, more especially 1, 2 or 3, of the hydrogen atoms in said moiety are replaced independently of each other by the corresponding number of substituents. The term “optionally substituted” as used herein includes substituted or unsubstituted. It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Substituents may suitably include halogen atoms and halomethyl groups such as CF₃ and CCl₃; oxygen containing groups such as oxo, hydroxy, carboxy, carboxyalkyl, alkoxy, alkoyl, alkoyloxy, aryloxy, aryloyl and aryloyloxy; nitrogen containing groups such as amino, alkylamino, dialkylamino, cyano, azide and nitro; sulfur containing groups such as thiol, alkylthiol, sulfonyl and sulfoxide; heterocyclic groups which may themselves be substituted; alkyl groups, which may themselves be substituted; and aryl groups, which may themselves be substituted, such as phenyl and substituted phenyl. Alkyl includes substituted and unsubstituted benzyl.

Where two or more moieties are described as being “each independently” selected from a list of atoms or groups, this means that the moieties may be the same or different. The identity of each moiety is therefore independent of the identities of the one or more other moieties.

Step (a) of the method described herein comprises providing a complex of a saccharide with an aryl boronate compound of formula (I):

Fp-L¹-Z-L²-Ar—B(OH)₂ (I)

wherein: Fp comprises a fluorophore; L¹ and L² are linker groups; Z is a fluorescence switch group; and Ar is optionally substituted aryl.

The group Fp comprises, consists essentially of, or consists of a fluorophore. The term “fluorophore” is used in its usual sense of a moiety that re-emits light in the visible or near infrared region upon excitation by light of shorter wavelength in the UV or visible region. The choice of fluorophore moiety Fp is not limited; any fluorophore moiety is suitable provided that its fluorescence is at least partially quenched in the free compound of formula (I) via the fluorescence switch group Z.

Representative fluorophores Fp include: Xanthene derivatives such as fluorescein, rhodamine, Oregon green, eosin, and Texas red; Cyanine and derivatives thereof such as indocarbocyanine, oxacarbocyanine, thiacarbocyanine, and merocyanine; Naphthalene derivatives such as dansyl and prodan derivatives; Coumarin derivatives; oxadiazole derivatives such as pyridyloxazole, nitrobenzoxadiazole and benzoxadiazole; Anthracene derivatives; anthraquinones; Pyrene derivatives such as cascade blue; Oxazine derivatives such as Nile red, Nile blue, cresyl violet, and oxazine 170; Acridine derivatives such as proflavin, acridine orange, acridine yellow; Arylmethine derivatives such as auramine, crystal violet, malachite green; and Tetrapyrrole derivatives such as porphin, phthalocyanine and bilirubin.

The fluorophore moiety Fp may suitably be substituted with one or more functional groups for improving water solubility of the compound of formula (I) and/or for attaching the compound of Formula (I) to a solid support. Suitable substituent groups for the fluorophore are groups R¹ as defined below in relation to the fluorophore of Formula (III).

Suitably, the fluorophore group Fp is an N-substituted-1,8-naphthalimide of general Formula (III):

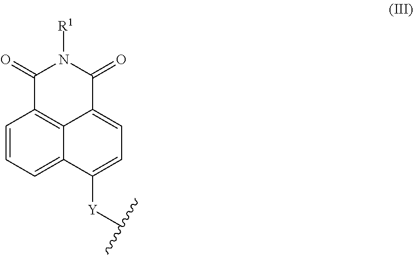

In Formula (III), R¹ is suitably hydrophilic for improving solubility of the compound in water, and/or R¹ suitably comprises a functional group suitable for attaching the moiety to a solid support e.g. by formation of an ester or amide linkage to the solid support. The substituent may therefore be a terminal substituent on R¹. For example, R¹ is suitably a (hetero) alkyl group bearing at least one hydrophilic substituent such as hydroxyl, carboxylate, sulfonate, phosphonate, amine or quaternary ammonium. Suitably, R¹ comprises from about 2 to about 30 contiguous atoms in the chain, more suitably from about 5 to about 15 such atoms. In embodiments, R¹ is a hydroxyalkyloxyalkyl group, for example it may be hydroxyethyloxyethyl.

The group Y in Formula (III) is suitably O, S or NR², where R² is suitably H or C1-C7 alkyl.

The naphthalene ring in Formula (III) is optionally further substituted at the 2,3,5,6 and/or 7 position.

The fluorescence switch group Z in the compounds of Formula (I) is a group having a lone pair of electrons for quenching the fluorescence of the fluorophore Fp by photo electron energy transfer (PET). For example, Z may be O, S or an amine group. Suitably, Z is an amine group NR³, wherein R³ is suitably H or C1-C7 alkyl, for example R³ may be H, methyl or ethyl.

More suitably, R¹ is a hydroxyalkyloxyalkyl group, Z is an amine group NR³ wherein R³ is H or C1-C7 alkyl, and the naphthalene rings are otherwise unsubstituted.

The switch Z is bonded through linker group L² to the arylboronic acid group. Suitably the linker group L² is optionally substituted (hetero)alkylene or (hetero)alkenylene having from 1 to 5 contiguous atoms, preferably 1 or 2 contiguous atoms, most preferably 1 carbon atom. More suitably it is C1-C3 alkylene, in particular methylene, whereby L² forms with the boronic acid group and the aryl group and the saccharide a ring structure in which the switch Z is inhibited by hydrogen bonding to the lone pair from quenching the fluorophore Fp.

The switch Z is bonded through linker group L¹ to the fluorophore Fp. Suitably the linker group L¹ is optionally substituted (hetero)alkylene or (hetero)alkenylene having from 1 to 5 contiguous atoms, preferably 1 to 3 contiguous atoms. More suitably it is C1-C3 alkylene, in particular ethylene, whereby L¹ retains the switch Z in sufficient proximity to the fluorophore Fp for the switch Z to quench fluorescence of the fluorophore Fp by PET.

The aryl group Ar suitably consists of one aryl ring, or two fused aryl rings. Most suitably it is phenyl. Suitably, the boronic acid group and the linker L¹ are located ortho on the aryl group. The aryl group may optionally be substituted at one or more other positions on the ring.

Accordingly, it can be seen that the compound of formula (I) suitably has the following structure (IV):

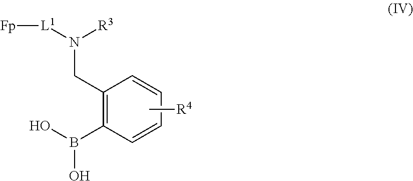

Or

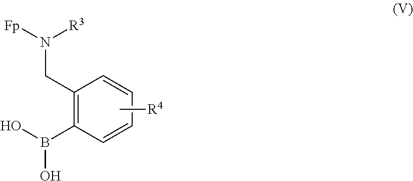

Or more suitably the following structure (VI):

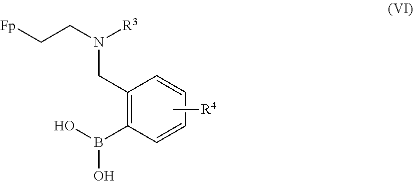

where R³ and are as defined above, and R⁴ indicates that the phenyl group is optionally substituted at one or more further positions. Most suitably, the phenyl group is not further substituted, i.e. R⁴ is H.

The compound of Formula (I) is reacted with a saccharide to form the boronate-saccharide complex. The saccharide is suitably a monosaccharide or a disaccharide. For example, the saccharide may be fructose, glucose, galactose, mannose or sucrose. Suitably, the saccharide is fructose. The saccharide is reacted with the compound of formula (I) in solution (or by contacting a solution of the saccharide with the compound of formula (I) immobilized on a solid support), typically at ambient temperatures such as about 10-50° C. The saccharide is suitably present in large molar excess, for example at least about 10×, 100× or 1000× molar excess, since excess saccharide present in solution does not interfere with the assay. The concentration of the saccharide solution is not critical. It may, for example, be from about 10 mM to about 1000 mM, for example from about 20 mM to about 250 mM.

Step (b) comprises contacting said aryl boronate-saccharide complex with said sample, whereby peroxynitrite in said sample cleaves said boronate-saccharide complex to produce a compound of formula (II):

Fp-L¹-Z-L²-Ar—OH (II)

The reaction with peroxynitrite is rapid and quantitative when performed in solution at ambient temperatures of about 10-50° C. The amount of the complex used for the measurement is suitably selected so that substantially all of the peroxynitrite, but not all of the complex is consumed by the reaction, thereby allowing the total peroxynitrite in the sample to be determined by reference to standard curves such as that shown in FIG. 4.

Step (c) comprises detecting a decrease in a fluorescence intensity of the fluorophore resulting from the cleavage reaction in step (b). The cleavage reaction with peroxynitrite frees the fluorescence switch group Z to quench fluorescence of the fluorophore Fp, whereby the product compound (II) exhibits lower fluorescence intensity than the complex. The product compound (II) may also exhibit even lower fluorescence intensity than the free starting compound (I). Fluorescence intensity can be measured with conventional fluorescence meters in conventional fashion.

It is envisaged that the present disclosure will have many applications in the fields of medical research, diagnosis and therapy. For example the disclosure could be used to evaluate new drugs which change the levels of peroxynitrite activity, in particular inhibitors which reduce peroxynitrite activity. Such inhibitor drugs could lead to novel treatments for diseases—such as Alzheimer's disease, Parkinson's disease, Huntington's disease, traumatic brain injury, and skeletal muscle hypertrophy. By way of further example, inhibitor drugs could lead to novel treatments for diseases—such as Alzheimer's disease, Parkinson's disease, Huntington's disease, amyotrophic lateral sclerosis, viral myocarditis, septic shock, cardiac allograft, transplant coronary artery disease, idiopathic dilated cardiomyopathy, atrial fibrillation, hypercholesterolemia, atherosclerosis, hypertension, diabetes (including Type 1 diabetes mellitus and Type 2 diabetes mellitus), diabetic nephropathy, traumatic brain injury and skeletal muscle hypertrophy.

The present disclosure could also be used to evaluate peroxynitrite activity in biological systems in pharmacological and biochemical experiments, thereby helping to elucidate the exact role of peroxynitrite in diseases related to endogenous peroxynitrite production as identified above—such as Alzheimer's disease, Parkinson's disease, Huntington's disease, traumatic brain injury, and skeletal muscle hypertrophy. A particularly promising application of the present disclosure will be for diagnostic measurement of peroxynitrite levels in patients for the detection or monitoring of disease states such as these. In these embodiments, the test sample can be a biological sample removed from a subject.

As used herein, the term “subject” refers to any animal, including, but not limited to, mammals, humans, non-human primates, rodents, and the like.

There is also provided a method for diagnosing or detecting a disease in a subject, including measuring the activity of peroxynitrite as described herein in a biological sample removed from the subject.

The term “diagnosing” encompasses both detection and identification of a medical condition and can include monitoring the progress of the said condition, for example following treatment. The methods of diagnosis therefore may involve taking a plurality of biological samples at intervals of time to monitor the progress of a medical condition. For example, the plurality of samples may be taken at intervals of from about 1 hour to about 1 year, suitably at intervals of from about 1 day to about 30 days.

The biological sample may suitably be sourced from a specimen of any body tissue or excretion or exudate. Samples include biological fluids—such as blood, plasma, serum and the like; organ or tissue or cell culture derived fluids; and fluids extracted from physiological tissues. Also included in the term are derivatives and fractions of such fluids. The cells may be dissociated, in the case of solid tissues, or tissue sections may be analysed. Alternatively, a lysate of the cells may be prepared for the purpose of a screening assay. Cell homogenates, differential centrifugation, cellular fractions/extracts, differentiation through the use of protease inhibitors, immunoblotting and enzyme assays and the like may also be employed. The sample may be subjected to further conventional preparation processes, in particular to separation or stabilization processes such as maceration, filtration, ultrafiltration, centrifugation, buffering, dilution, etc. The sample may be of or may be derived from lymph nodes or lymph fluid.

The sample can be compared with a reference standard from another subject. The reference standard may be established by prospective and/or retrospective statistical studies. Healthy subjects who have no clinically evident disease or abnormalities may be selected for statistical studies. Diagnosis may be made by the finding of statistically significant different levels of peroxynitrite compared to the reference standard.

If the measured amount or concentration of peroxynitrite is higher than that of the reference standard, the amount or concentration of peroxynitrite may be considered to be increased. If the amount or concentration of peroxynitrite is lower than that of the reference standard, the amount or concentration of peroxynitrite may be considered to be decreased. If the amount or concentration of peroxynitrite is higher or lower than that of the reference standard then the amount or concentration of peroxynitrite in the sample will be considered to be abnormal. If the amount or concentration of peroxynitrite is increased as compared to the reference standard then this sample is indicative of the presence of disease such as those identified above.

Suitably the reference standard is age matched to the subject. Suitably the reference standard is ethnicity matched to the subject. The reference standard need not necessarily involve the parallel testing of another sample alongside the testing of the sample from the subject of interest. The reference standard may in fact be a numerical value determined on a past occasion. Therefore the comparison with the reference standard may be a purely numerical exercise of comparing the two determined values. When testing samples from the same subject, the samples may be obtained repeatedly, such as daily, every two or three days, weekly, fortnightly or at longer intervals.

Suitably, the change in the amount or concentration of peroxynitrite is a significant change. In certain embodiments, a significant increase is one where the amount or concentration of peroxynitrite in the sample obtained from the subject is more than 10%, 20%, 30%, 40%, 50%, 60%, 70%, 80%, 90%, 100%, 200%, 300%, 400%, 500%, 600%, 700%, 800%, 900% or 1000% higher as compared to the corresponding level in the sample obtained from the reference standard. In certain embodiments, a significant increase means that the increase is significant using the criteria p<0.05, 2-tailed test.

The methods described herein are expected to be especially useful for detecting or diagnosing a disease related to peroxynitrite activity—such as those identified above.

Suitably, the method further comprises a reference measurement to enable appropriate corrections to normalise the peroxynitrite in the biological sample to compensate for variations in dilution, etc. For example, the method may further comprise measuring a total polypeptide content of the test sample and normalising the measured level of peroxynitrite to the total polypeptide content. This allows variation in the composition of the test sample to be corrected. For example, total polypeptide content can be determined using the Bradford polypeptide assay (Bradford M M, Anal. Biochem. 1976, 72:248-254).

In embodiments, the method is performed to detect peroxynitrite production in a living cell. The cell can be studied in a suitable medium following removal from the human or animal body. The compound of Formula (I) and saccharide, and/or complexes of the compound of Formula (I) and saccharide are low molecular weight substances that are rapidly internalized into cells. This allows individual cells where peroxynitrite is being produced more rapidly to be identified, since these cells will show a more rapid decrease in fluorescence intensity that can be observed with fluorescence microscopy. Examples of such imaging methods are described below.

In these and other embodiments, the method may be used to image or visualise a spatial distribution of peroxynitrite. These embodiments could be especially useful for identifying tissues or individual cells that are producing peroxynitrite in vivo or in vitro at elevated rates.

A further aspect relates to a method for identifying a compound that modulates the production of peroxynitrite by living cells comprising the steps of: (i) providing a sample comprising living cells; (ii) providing at least one test compound; and (iii) comparing the rates of peroxynitrite production of said sample in the presence and absence of said test compound by detecting said peroxynitrite by a method described herein, wherein a change in the rate or production of peroxynitrite in the presence and absence of said test compound is indicative that said test compound modulates the production of peroxynitrite.

For example, the rate of production of peroxynitrite may be compared by comparing the rate of quenching of the fluorescence of the complex internalised into the cell cytoplasm by fluorescence microscopy as described herein.

A test compound that is subjected to screening may be any compound of interest and includes small organic compounds, polypeptides, peptides, higher molecular weight carbohydrates, polynucleotides, fatty acids and lipids, and the like. Test compounds may be screened individually or in sets or combinatorial libraries of compounds. Test compounds can be obtained from a wide variety of sources including libraries of synthetic or natural compounds. Libraries of natural compounds in the form of bacterial, fungal, plant and animal extracts can be used. Natural or synthetically produced libraries and compounds that are modified through conventional chemical, physical and biochemical means may be used to produce combinatorial libraries. Known pharmacological agents may be subjected to directed or random chemical modifications, such as acylation, alkylation, esterification, acidification to produce structural analogues for screening.

When screening using a combinatorial library, a large library of chemically similar or diverse molecules can be screened. In one approach combinatorial synthesis is employed to prepare a diverse set of molecules in which several components predicted to be associated with the modulation of peroxynitrite are systematically varied. In combinatorial screening, the number of hits discovered is proportional to the number of molecules tested. The large numbers of compounds, which may reach thousands of compounds tested per day, can be screened using a suitable high throughput screening technique, in which laboratory automation and robotics may be applied. Examples of methods for the synthesis of molecular libraries can be found in the art, for example in: Proc. Natl. Acad. Sci. U.S.A. 90:6909 (1993); Proc. Nad. Acad. Sci. USA 91:11422 (1994); J. Med. Chem. 37:2678 (1994); Science 261:1303 (1993); Angew. Chem. Int. Ed. Engl. 33.2059 (1994); Angew. Chem. Int. Ed. Engl. 33:2061 (1994); and J. Med. Chem. 37:1233 (1994). Libraries of compounds may be presented in solution (see, for example, Biotechniques 13:412-421 (1992)), or on beads (Nature 354:82-84 (1991)), chips (Nature 364:555-556 (1993)), bacteria or spores (U.S. Pat. No. 5,223,409), plasmids (Proc. Nad. Acad. Sci. USA 89:18651869 (1992)) or on phage (Science 249:386-390 (1990); Science 249:404-406 (1990); Proc. Natl. Acad. Sci. 87:6378-6382 (1990); J. Mol. Biol. 222:301 (1991)).

A small organic compound includes a compound of molecular weight less than about 5000, usually less than about 2500, usually, less than about 2000, more usually, less than about 1500, preferably about 100 to about 1000. The small organic compounds may be either biological or synthetic organic compounds. The atoms present in the small organic compound are generally in the group comprising carbon, hydrogen, oxygen, and nitrogen and may include halogens, boron, phosphorus, selenium and sulfur if in a pharmaceutically acceptable form. Generally, oxygen, nitrogen, sulfur or phosphorus, if present, are bound to carbon or one or more of each other or to hydrogen to form various functional groups such as, for example, carboxylic acids, alcohols, thiols, carboxamides, carbamates, carboxylic acid esters, amides, ethers, thioethers, thioesters, phosphates, phosphonates, olefins, ketones, amines, aldehydes, and the like. The small organic compounds, as the term is used herein, also include small peptides, small oligonucleotides, small polysaccharides, fatty acids, lipids, and the like having a molecular weight less than about 5000.

Polypeptides that have a molecular weight of at least about 5,000, more usually at least about 10,000 can be screened. The test polypeptides will generally be from about 5,000 to about 5,000,000 or more molecular weight, more usually from about 20,000 to about 1,000,000 molecular weight. A wide variety of polypeptides may be considered such as a family of polypeptides having similar structural features, polypeptides having particular biological functions, polypeptides related to specific microorganisms, particularly disease causing microorganisms. Such polypeptides include cytokines or interleukins, enzymes, protamines, histones, albumins, immunoglobulins, scleropolypeptides, phosphopolypeptides, mucopolypeptides, chromopolypeptides, lipopolypeptides, nucleopolypeptides, glycopolypeptides, T-cell receptors, proteoglycans, somatotropin, prolactin, insulin, pepsin, polypeptides found in human plasma, blood clotting factors, blood typing factors, polypeptide hormones, cancer antigens, tissue specific antigens, peptide hormones, nutritional markers, tissue specific antigens, and synthetic peptides, which may or may not be glycated.

Polynucleotides can be screened. The test polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also contemplated. The higher molecular weight polynucleotides can have from about 20 to about 5,000,000 or more nucleotides.

In another aspect, there is provided a kit for detecting peroxynitrite by a method according to the invention, comprising either (i) an arylboronate of Formula (I) and a saccharide in separately packaged form, or (ii) a complex of an arylboronate of Formula (I) and a saccharide in packaged form. In either case, the arylboronate or complex thereof may be immobilized on a solid support, for example by forming a covalent bond between the support and a suitable functionalized substituent on the fluorophore moiety as described above. In either case, the packaged components are suitably sterile and packaged in microorganism-impermeable containers.

In another aspect, the disclosure provides an arylboronate of Formula (I) and a saccharide for combined use in a method according to the invention.

In another aspect, there is provided an arylboronate of Formula (I), or a complex of an arylboronate of Formula (I) and a saccharide, for use in a method according to the present invention.

Specific embodiments of the invention will now be described further with reference to the following non-limiting examples.

### EXAMPLES

### General Methods

All the chemicals were purchased from Sigma-Aldrich Chemical Co., and used as received.

Nitric oxide (NO) was prepared by treating sulfuric acid (3.6 M) solution with sodium nitrite solution (7.3 M) and its stock solution (2.0 mM) was prepared by bubbling NO into deoxygenated deionized water for 30 min;

ROO. was generated from 2,2′-azobis (2-amidinopropane) dihydrochloride. AAPH (2,2′-azobis (2-amidinopropane) dihydrochloride, 1 M) was added into deionizer water, and then stirred at 37° C. for 30 min;

Superoxide was generated from KO₂ with a saturated solution of KO₂ in DMSO (˜1 mM);

Hydroxyl radical was generated by Fenton reaction. To prepare .OH solution, hydrogen peroxide (H₂O₂) was added in the presence of Fe(ClO₄)₂ (Abo, M.; Urano, Y.; Hanaoka, K.; Terai, T.; Komatsu, T.; Nagano, T. J. Am. Chem. Soc. 2011, 133, 10629).

Peroxynitrite solution was synthesized as reported (Reed, J. W.; Ho, H. H.; Jolly, W. L. J. Am. Chem. Soc. 1974, 96, 1248). The concentration of peroxynitrite was estimated by using extinction co-efficient of 1670 cm⁻¹ M⁻¹ at 302 nm in 0.1 N sodium hydroxide aqueous solutions.

The concentration of hypochlorite (⁻OCl) was determined from the absorption at 292 nm (ε=350 M⁻¹ cm⁻¹).

The concentration of hydrogen peroxide (H₂O₂) was determined from the absorption at 240 nm (ε=43.6 M⁻¹ cm⁻¹).

All other chemicals were from commercial sources and of analytical reagent grade, unless indicated otherwise. The fluorescent titrations with peroxynitrite were carried out at 25° C. in pH 7.30 PBS buffer (KH₂PO₄, 1/15 M; Na₂HPO₄, 1/15 M) and pH 5.00 buffer (NaOAc/HOAc, 50 mM).

The saccharide-boronic acid complexes were formed by mixing free boronic acid (2 μM) with D-fructose (100 mM) for 10 min in situ.

### Chemical Synthesis

Boronate probe 1 (λ_{abs}=440 nm, ε=9500 M⁻¹ cm⁻¹) was prepared according to the following scheme:

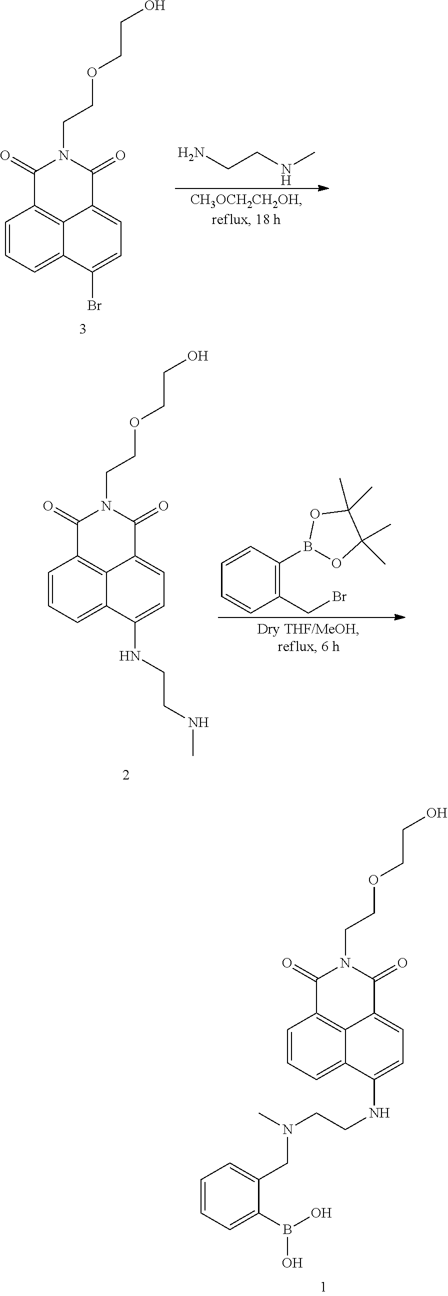

### Synthesis of Compound 3

Under a nitrogen atmosphere, to a solution of 4-bromo-1, 8-naphthalic anhydride (0.30 g, 1.08 mmol) in anhydrous ethanol (20 mL) was added 2-(2-aminoethoxy) ethanol (0.12 g, 1.19 mmol). The mixture was heated at reflux for 3 h and concentrated under reduced pressure. Purified by flash chromatography (CH₂Cl₂:CH₃OH, 50:1, v/v) afforded a yellow powder (0.35 g). Yield: 88%. Mp: 122-124° C. ¹H NMR (300 MHz, CDCl₃) δ 8.60 (d, J=7.5 Hz, 1H), 8.53 (d, J=8.4 Hz, 1H), 8.36 (d, J=7.8 Hz, 1H), 7.98 (d, J=7.8 Hz, 1H), 7.79 (dd, J₁=8.4 Hz, J₂=7.2 Hz, 1H), 4.38 (t, J₁=J₂=5.4 Hz, 2H), 3.79 (t, J₁=J₂=5.7 Hz, 2H), 3.59-3.63 (m, 4H); ¹³C NMR (75 MHz, CDCl₃), δ 163.9, 163.8, 133.4, 132.2, 131.4, 131.1, 130.5, 128.9, 128.1, 122.8, 121.9, 72.3, 68.3, 61.8, 39.6; HRMS (ES+) m/z calcd for [M+Na]⁺, 386.0004. found, 386.0028.

### Synthesis of Compound 2

A solution of 2-bromoethylamine hydrobromide (1.5 g, 4.12 mmol) and N-methylethylene-diamine (0.322 g, 95% wt., 4.12 mmol) in 2-methoxyethanol was refluxed overnight under a nitrogen atmosphere. The mixture was concentrated under reduced pressure to obtain a purple oil product. Column chromatography on silica gel (CH₂Cl₂:CH₃OH, 3:1, v/v) was employed to purify the crude product as yellow solid (0.8 g). Yield: 55%. ¹H NMR (300 MHz, CDCl₃) δ 8.49 (dd, J₁=7.5 Hz, J₂=0.9 Hz 1H), 8.39 (d, J=8.4 Hz, 1H), 8.19 (dd, J₁=8.7 Hz, J₂=0.9 Hz 1H), 7.55 (dd, J₁=8.4 Hz, J₂=7.5 Hz, 1H), 6.59 (d, J=8.4 Hz, 1H), 6.39 (br s, 1H), 4.41 (t, J₁=J₂=5.4 Hz, 2H), 3.87 (t, J₁=₂=5.4 Hz, 2H), 3.72 (m, 4H), 3.48 (m, 2H), 3.07 (t, J₁=₂=6.0 Hz, 2H), 2.54 (s, 3H); ¹³C NMR (75 MHz, CDCl₃), δ 165.0, 164.5, 150.0, 134.6, 131.2, 129.6, 126.9, 124.4, 122.3, 120.2, 109.4, 104.1, 72.4, 68.8, 61.7, 50.8, 42.1, 39.3, 35.9; FIRMS (ES+) Calcd for ([M+H])⁺, 358.1766. Found, 358.1772.

### Synthesis of Compound 1

The product compound 1 was synthesised by refluxing compound 2 (0.20 g, 0.56 mmol) with 2-bromomethylphenylboronic acid pinacol ester (0.28 g, 0.94 mmol) in dry tetrahydrofuran (20 mL) for 6 hours. The product was purified on silica gel, using dichloromethane/methanol 5:1 to afford 1 as yellow oil (0.07 g). Yield: 25%. ¹H NMR (300 MHz, CD₃OD) δ 8.60 (d, J=8.4 Hz, 1H), 8.44 (d, J=7.2 Hz, 1H), 8.28 (d, J=8.4 Hz, 1H), 7.58 (dd, J₁=J₂=8.1 Hz, 2H), 7.22 (m, 3H), 6.80 (d, J=8.4 Hz, 1H), 4.32 (t, J₁=₂=6.0 Hz, 2H), 4.17 (s, 2H), 3.74-3.84 (m, 4H), 3.57-3.63 (m, 4H); 3.29-3.34 (m, 2H), 2.59 (s, 3H); ¹³C NMR (75 MHz, CD₃OD), δ 166.6, 166.1, 152.4, 136.2, 134.6, 132.6, 131.5, 130.3, 128.8, 128.6, 128.5, 125.9, 123.6, 122.4, 110.5, 105.5, 73.8, 69.6, 64.7, 62.6, 56.3, 41.7, 40.5, 39.8; HRMS (ES+) m/z calcd for [M+H]⁺, 492.2305. found, 492.2326.

### Experimental Measurements

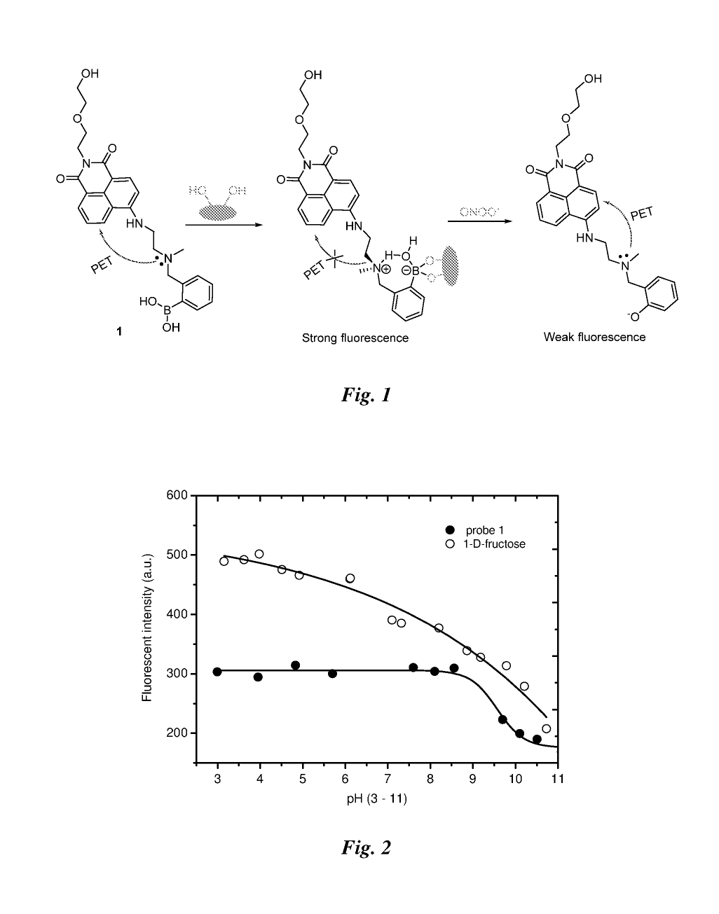

The overall reaction scheme of probe 1 with a saccharide to form a fluorescent complex, with subsequent cleavage of the complex to form a phenol in which fluorescence is quenched by photoelectron energy transfer (PET) is shown in FIG. 1.

pH Titration.

A pH titration was carried out to evaluate the pH effect on the fluorescence of probe 1 (2 μM). As shown by FIG. 2, the fluorescence intensity of the probe 1 decreased at pH values above 8.0. While, in the range between 3.0 and 8.0, the pH change had minimal effect on the fluorescence intensity. Thus, the probe can be expected to work well under physiological conditions (pH 7.30, PBS buffer). In the presence of D-fructose (100 mM), the fluorescence of the formed 1-D-fructose complex increases due to the enhanced N—B interaction at different pH values. The fluorescence of 1-D-fructose complex decreases over a pH range of 3-11.

UV-Vis and Emission Spectra and Reaction with ONOO⁻.

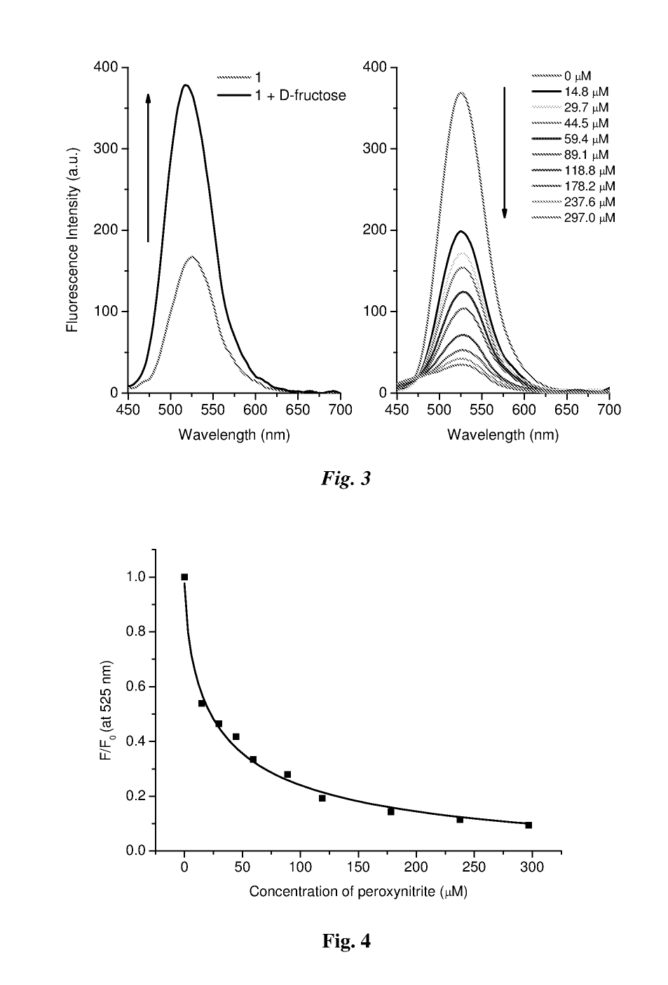

In the case of probe 1, a significant “off-on” signal response is seen on binding with D-fructose due to an inhibition of PET mechanism. From FIG. 3a, the maximum fluorescence intensity of probe 1 (2 μM, λₑₘ=525 nm) is increased two-fold in the presence of D-fructose (100 mM) at pH 7.3 buffer solution.

However, when the arylboronic ester moiety of probe 1 was transformed into a phenol upon adding peroxynitrite, the fluorescence was further reduced due to the stronger PET from the amine in the boron free system as shown in FIG. 3b. This is plotted in FIG. 4 as a dose-dependent titration curve, from which it can be seen that the enhanced fluorescence of probe 1-D-fructose complex was finally reduced to a F (in the presence of ONOO⁻)/F₀ (in the absence of ONOO⁻)=ca. 0.10 over a concentration range of ONOO⁻ (0-297 μM).

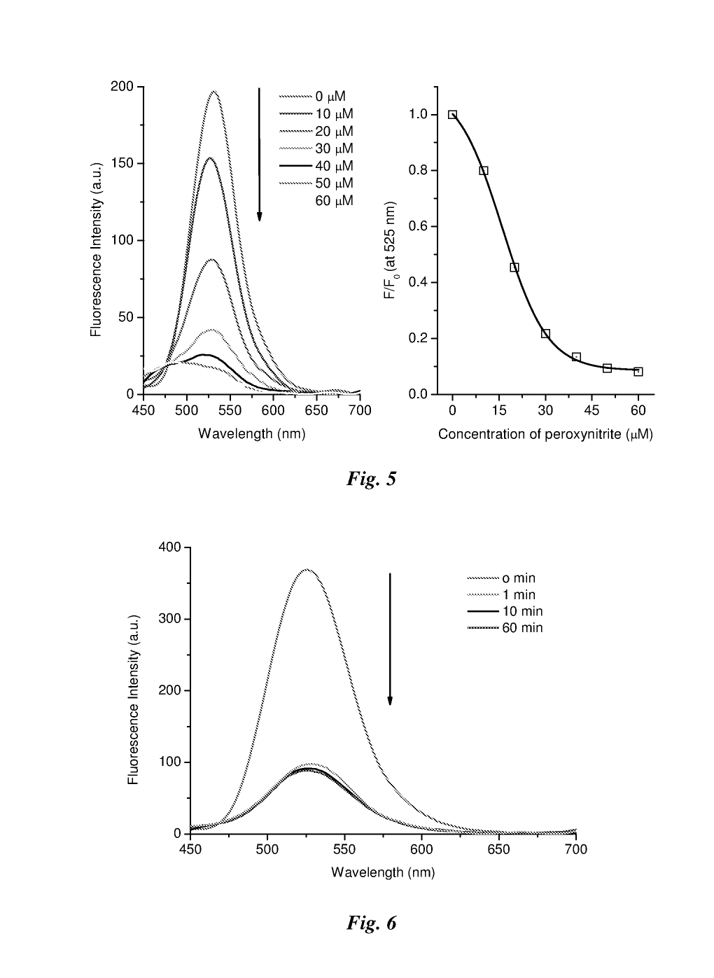

The effect of peroxynitrite of the free probe 1 in the absence of saccharide was also studied. It was found that, for the saccharide free system, small amounts of peroxynitrite (60 μM) caused a big change in fluorescence intensity F/F₀=ca. 0.10 (FIGS. 5a, 5b).

In the UV-Vis spectra, the free boronic acid probe 1 (2 μM) displayed a maximum absorption (A=0.026) at 440 nm while the binding of D-fructose (100 mM) led to a decrease to A=0.019 at the maximum absorption wavelength. While the presence of ONOO⁻ (100 μM), a new peak (A=0.021) at 360 nm developed due to the formation of phenol.

FIG. 6 shows time-course kinetic measurement of the fluorescence response of probe 1-D-fructose (probe 1, 2 μM; D-fructose, 100 mM) to peroxynitrite (100 μM) at pH 7.30 buffer solution. The spectra were collected with excitation at 410 nm (Ex slit: 5.0, Em slit: 5.0). It can be seen that the reaction with peroxynitrite is essentially complete after only about 1 minute.

Thus, the fluorescence of probe 1 is turned on by saccharide binding, since boronic ester formation causes an enhanced interaction between the neighboring amine and the boron atom. This NB interaction also hampers the reaction between boron and peroxynitrite in the presence of saccharides resulting in a slower decrease in the observed fluorescence.

Emission Spectra in the Presence of H₂O₂.

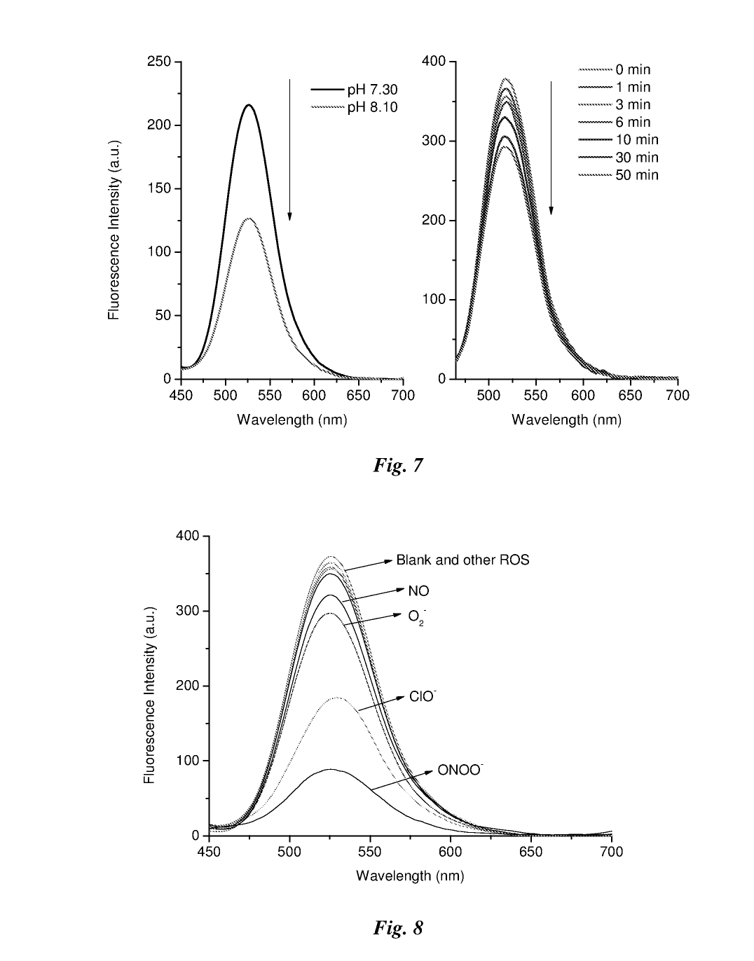

Since boronate-based derivatives can be oxidized to phenol by H₂O₂ and ONOO⁻, it is important to discriminate them by fluorescence tools. Independently, the present inventors tested the responses of probe 1 and 1-D-fructose complex towards hydrogen peroxide (FIG. 7). With the free boronic acid system, the fluorescence of probe 1 (2 μM) increased to F/F₀=ca. 1.59 in the presence of hydrogen peroxide (100 μM) over 1 h at pH 7.30 buffer solution. When the solution was adjusted to pH 8.10, the fluorescence decreased most probably due to the decomposition of the intermediate to phenol (FIG. 7a). This is different from the process observed for the detection of ONOO⁻ in which boronic acid was transformed into phenol quickly and directly. While in the case of the 1-D-fructose complex, the fluorescence showed only a slight drop F/F₀=ca.0.75 even after the addition of H₂O₂ (1 mM) over 1 h (FIG. 3b).

Therefore, the probe 1-D-fructose complex does not produce a significant response to H₂O₂. Indeed, the connection of probe 1 with D-fructose not only strengthens the fluorescence signal, but also protects the boronic acid to oxidation by hydrogen peroxide via the N—B interaction. However, with peroxynitrite, the oxidation reaction between boron and peroxynitrite was still very rapid and is complete within 1 min.

As already noted, the sugar complex reacts stoichiometrically and rapidly with ONOO⁻ to form the phenol product. Therefore, under conditions generating both H₂O₂ and ONOO⁻, the probe 1-D-fructose complex preferentially reacts with ONOO⁻.

Selectivity Tests Towards ROS/RNS.

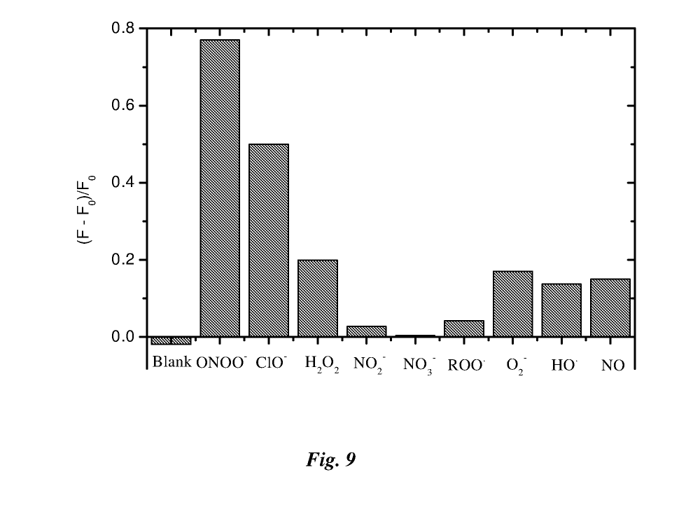

The selectivity of the 1-D-fructose complex was also studied towards other reactive oxygen and nitrogen species, such as hypochlorite (⁻OCl), nitric oxide (NO), nitrite (NO₂⁻), nitrate (NO₃⁻), peroxyl radical (ROO.), superoxide (O₂⁻), hydroxyl radical (.OH) in pH 7.30 buffer solution (FIG. 8). Among them, only hypochlorite (100 μM) caused a big fluorescence decrease (F−F₀)/F=ca. 0.48 over 1 h (FIG. 9). As reported previously, aryl boronic acid and ester can be oxidized into phenol by hypochlorite. However, under the same concentrations of ⁻OCl (100 μM) and ONOO⁻ (100 μM), peroxynitrite reacts much more strongly with the 1-D-fructose sensing system ((F−F₀)/F=ca. 0.78).

The fluorescence reaction of 1-D-fructose complex towards ONOO⁻ amd H₂O₂ at pH 5.0 buffer solution in order to mimic the acidic conditions found in cancer cells (FS5) was also studied. Under the same concentrations of H₂O₂ (500 μM) and ONOO⁻ (500 μM), the fluorescence ratio only slightly decreased to (F−F₀)/F=ca. 0.09 with H₂O₂ while the value decreased significantly to (F−F₀)/F=ca. 0.65 for ONOO⁻. Therefore, the fluorescent probe can be employed for the selective and sensitive detection of peroxynitrite under physiological and pathological conditions which are either low or have no ⁻OCl.

Intracellular Imaging for Exogenous and Endogenous ONOO⁻.

The sensor has the potential ability for exogenous and endogenous peroxynitrite cell imaging, such as Hela cells (human epithelial adenocarcinoma) and RAW 264.7 cells (mouse macrophage cell).

The above examples have been described for the purpose of illustration only. Many other embodiments of the compounds and methods of the invention falling within the scope of the accompanying claims will be apparent to the skilled reader.

Any publication cited or described herein provides relevant information disclosed prior to the filing date of the present application. Statements herein are not to be construed as an admission that the inventors are not entitled to antedate such disclosures. All publications mentioned in the above specification are herein incorporated by reference. Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry and biology or related fields are intended to be within the scope of the following claims.

1. A method of detecting peroxynitrite in a sample comprising the steps of:
(a) providing a complex of a saccharide with an aryl boronate compound of formula (I):
Fp-L¹-Z-L²-Ar—B(OH)₂ (I)
wherein:
Fp comprises a fluorophore;
L¹ and L² are linker groups;
Z is a fluorescence switch group; and
Ar is optionally substituted aryl;
(b) contacting said aryl boronate-saccharide complex with said sample, whereby peroxynitrite in said sample cleaves said boronate-saccharide complex to produce a compound of formula (II):
Fp-L¹-Z-L²-Ar—OH (II)
and
(c) detecting a decrease in a fluorescence intensity of said fluorophore resulting from said cleavage reaction in step (b).

2. The method according to claim 1, wherein the method comprises quantitating an amount of peroxynitrite in said sample by comparing said decrease in fluorescence intensity with decreases observed for reference amounts or concentrations of peroxynitrite.

3. The method according to claim 1, wherein said method is performed on a sample of a biological fluid or tissue.

4. The method according to claim 3, wherein said sample of a biological fluid or tissue is a sample removed from a human or animal body, and said method further comprises comparing said decrease in fluorescence intensity with a threshold value indicative of a disease state.

5. The method according to claim 1, wherein said method is performed to detect peroxynitrite in a living cell.

6. The method according to any of claim 1, wherein said method is performed to image a spatial distribution of peroxynitrite.

7. The method according to claim 1, wherein said step (b) is performed at a pH of about 9 or less, preferably about 6 or less.

8. The method according to claim 1, wherein said step (a) comprises reacting said arylboronate of Formula (I) with a saccharide in solution to form a solution of said complex, and said step (b) comprises mixing said solution with said sample.

9. The method according to claim 1, wherein said Fp group is an N-substituted 1,8-naphthalimide of structure (III): 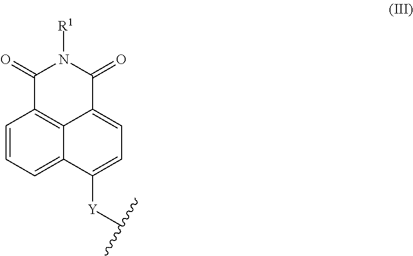
wherein: R¹ is a (hetero)alkyl group comprising from about 2 to about 30 contiguous atoms in the chain and bearing at least one hydrophilic substituent such as hydroxyl, carboxylate, sulfonate, phosphonate, amine or quaternary ammonium; and Y is O, S or NR², where R² is H or C1-C7 alkyl.

10. The method according to claim 1, wherein said Ar group is an optionally substituted phenyl or naphthyl group, and said L² and B(OH)₂ groups are ortho positioned on said phenyl group.

11. The method according to claim 1, wherein said fluorescence switch group is NR³, wherein R³ is H or optionally substituted C1-C7 alkyl, preferably methyl or ethyl.

12. The method according to claim 1, wherein L¹ and L² are independently selected from saturated or unsaturated, linear or branched aliphatic chains including 1-6 carbon atoms, preferably 1 or 2 carbon atoms.

13. The method according to claim 1, wherein said compound of Formula (I) has the following structure (IV): 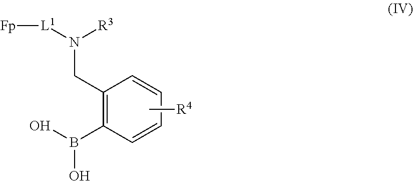
where R³ and L¹ are as defined above, and R⁴ indicates that the phenyl group is optionally substituted at one or more further positions.

14. The method according to claim 1, wherein said saccharide is a monosaccharide or a disaccharide, preferably D-fructose.

15. A kit for detecting peroxynitrite by a method according to claim 1, comprising either (i) an arylboronate of Formula (I) and a saccharide in separately packaged form, or (ii) a complex of an arylboronate of Formula (I) and a saccharide in packaged form.

16. An arylboronate of Formula (I) and a saccharide for combined use in a method according to claim 1.

17. An arylboronate of Formula (I), or a complex of an arylboronate of Formula (I) and a saccharide, for use in a method according to claim 1.

18. An arylboronate of Formula (I), or a complex of an arylboronate of Formula (I) and a saccharide, for use in a method of diagnosis of a disease state comprising the steps of: measuring the level of peroxynitrite in a sample of biological fluid or tissue removed from the human or animal body by a method according to claim 1, and comparing said level with a reference level of peroxynitrite.

19. The arylboronate of Formula (I), or a complex of an arylboronate of Formula (I) and a saccharide, as claimed in claim 18, wherein said disease state is selected from Alzheimer's disease, Parkinson's disease, Huntington's disease, amyotrophic lateral sclerosis, viral myocarditis, septic shock, cardiac allograft, transplant coronary artery disease, idiopathic dilated cardiomyopathy, atrial fibrillation, hypercholesterolemia, atherosclerosis, hypertension, diabetes (including Type 1 diabetes mellitus and Type 2 diabetes mellitus), diabetic nephropathy, traumatic brain injury and skeletal muscle hypertrophy.

20. A method for diagnosing and/or detecting a disease in a subject, comprising the steps of:
(i) providing a sample from a subject;
(ii) measuring a level of peroxynitrite in said sample according to the method of claim 1;
(iii) comparing the measurement from step (ii) with a reference standard; and
(iv) using said comparison from step (iii) to determine whether the subject has a disease;
(v) optionally, treating said disease;
(vi) optionally, repeating steps (i) to (iv) to monitor the progress of the disease.

21. The method according to claim 20, wherein said disease is selected from the group consisting of Alzheimer's disease, Parkinson's disease, Huntington's disease, amyotrophic lateral sclerosis, viral myocarditis, septic shock, cardiac allograft, transplant coronary artery disease, idiopathic dilated cardiomyopathy, atrial fibrillation, hypercholesterolemia, atherosclerosis, hypertension, diabetes (including Type 1 diabetes mellitus and Type 2 diabetes mellitus), diabetic nephropathy, traumatic brain injury and skeletal muscle hypertrophy.

22. A method for identifying a compound that modulates the production of peroxynitrite by living cells comprising the steps of:
(i) providing a sample comprising living cells;
(ii) providing at least one test compound;
(iii) comparing the rates of peroxynitrite production of said sample in the presence and absence of said test compound by detecting said peroxynitrite by a method according to claim 1,
wherein a change in the rate or production of peroxynitrite in the presence and absence of said test compound is indicative that said test compound modulates the production of peroxynitrite.

23. (canceled)
